(19) United States
(12) Patent Application Publication
(10) Pub. No.: US 2011/0022128 A1
(43) Pub. Date: Jan. 27, 2011

(54) HAIR-GROWTH DEVICE AND HAIR-GROWTH METHOD

(76) Inventors: Takehiro Nakagawa, Takatsuki-shi (JP); Chosei Hamada, Kadoma-shi (JP)

Correspondence Address: EDWARDS ANGELL PALMER & DODGE LLP, P.O. BOX 55874, BOSTON, MA, 02205, US

(21) Appl. No.: 12/736,343

(22) PCT Filed: Mar. 31, 2009
(86) PCT No.: PCT/JP2009/056668

§ 371 Date: Sep. 30, 2010

(30) Foreign Application Priority Data
Mar. 31, 2008 (JP) 2008-093584

(51) Int. Cl. A61N 5/06

(52) U.S. Cl. 607/88

(57) ABSTRACT

The hair-growth device (10) is configured to grow hair utilizing light. The hair-growth device (10) includes an irradiator (20) configured to irradiate one's skin with light. The irradiator (20) is configured to irradiate with the light having a wavelength equivalent to a specific absorption wavelength of water.

### TECHNICAL FIELD

The present invention is directed to hair-growth devices and hair-growth methods, and more particularly to a hair-growth device and a hair-growth method utilizing light for hair-growth.

### BACKGROUND ART

In the past, there has been a hair-growth method using a medical agent. Moreover, there has been a hair-growth method which grows hair by irradiating a skin with light having a wavelength of 890 nm (see Journal of the Korean Society Plastic & Reconstructive Surgeons 2004 pages 1 to 8).

The above mentioned hair-growth method using a medical agent or light irritates mildly a skin or skin inner tissue, thereby promoting hair-growth. Therefore, a user performing the above mentioned hair-growth method is likely to feel uncomfortable.

### DISCLOSURE OF INVENTION

In view of the above insufficiency, the present invention has been aimed to propose a hair-growth device and method which are capable of growing a user's hair in a comfortable manner.

The hair-growth device in accordance with the present invention includes an irradiator configured to irradiate one's skin with light having a wavelength equivalent to a specific absorption wavelength of water.

According to the invention; it is possible to provide hair-growth effect without using a medical agent. Additionally, the light having a wavelength equivalent to the specific absorption wavelength of water does not irritate a skin or skin inner tissue. Therefore, it is possible to grow a user's hair in a comfortable manner without injuring a skin.

In a preferred embodiment, the light has a wavelength of 950 nm or 1450 nm.

According to the preferred embodiment, it is possible to emit the light having a wavelength of 950 nm or 1450 nm, which is equivalent to the specific absorption wavelength of water and produces an excellent effect for improvement of the hair-growth effect.

In a preferred embodiment, the irradiator includes a first light source configured to emit light having a wavelength of 950 nm, a second light source configured to emit light having a wavelength of 1450 nm, and a controller configured to control the first light source and the second light source. The controller is configured to turn on the first light source and the second light source simultaneously or alternately.

According to this preferred embodiment, the light of a wavelength of 950 nm suitable for a man in his twenties and the light of a wavelength of 1450 nm suitable for a man in his fifties can be emitted simultaneously or alternately. Therefore, it is possible to provide the stable hair-growth effect irrespective of the user's age.

In a preferred embodiment, the hair-growth device includes a time controller configured to allow the irradiator to emit continuously the light for at least 15 minutes.

According to this preferred embodiment, it is possible to improve the hair-growth effect.

The hair-growth method in accordance with the present invention including a step of irradiating one's skin with light having a wavelength equivalent to a specific absorption wavelength of water.

According to the invention, it is possible to provide hair-growth effect without using a medical agent. Additionally, the light having a wavelength equivalent to the specific absorption wavelength of water does not irritate a skin or skin inner tissue. Therefore, it is possible to grow a user's hair in a comfortable manner without injuring a skin.

In a preferred embodiment, the light emitted to the skin has a wavelength of 950 nm or 1450 nm.

According to the preferred embodiment, it is possible to emit the light having a wavelength of 950 nm or 1450 nm, which is equivalent to the specific absorption wavelength of water and produces an excellent effect for improvement of the hair-growth effect.

In a preferred embodiment, first light having a wavelength of 950 nm and second light having a wavelength of 1450 nm are emitted simultaneously or alternately.

According to this preferred embodiment, it is possible to provide the stable hair-growth effect irrespective of a user's age.

In a preferred embodiment, the light is emitted to the skin continuously for at least 15 minutes.

According to this preferred embodiment, it is possible to improve the hair-growth effect.

In a preferred embodiment, the wavelength of the light emitted to the skin is selected on the basis of an age of a person to be irradiated with the light.

According to this preferred embodiment, it is possible to provide the hair-growth effect suitable for the age.

### BRIEF DESCRIPTION OF DRAWINGS

FIG. 1 is a schematic view illustrating a hair-growth device of one embodiment in accordance with the present invention,

FIG. 2 is a schematic view illustrating an irradiator of the above hair-growth device,

FIG. 3A is a graph showing a transition of the number of hairs,

FIG. 3B is a graph showing a transition of the number of hairs,

FIG. 4A is a graph showing a transition of a thickness of a hair,

FIG. 4B is a graph showing a transition of a thickness of a hair,

FIG. 5A is a graph showing a transition of a length of a hair,

FIG. 5B is a graph showing a transition of a length of a hair, and

FIG. 6 is a graph showing a relation between a hair-growth effect and an irradiation time of light of a wavelength equivalent to a specific absorption wavelength.

### BEST MODE FOR CARRYING OUT THE INVENTION

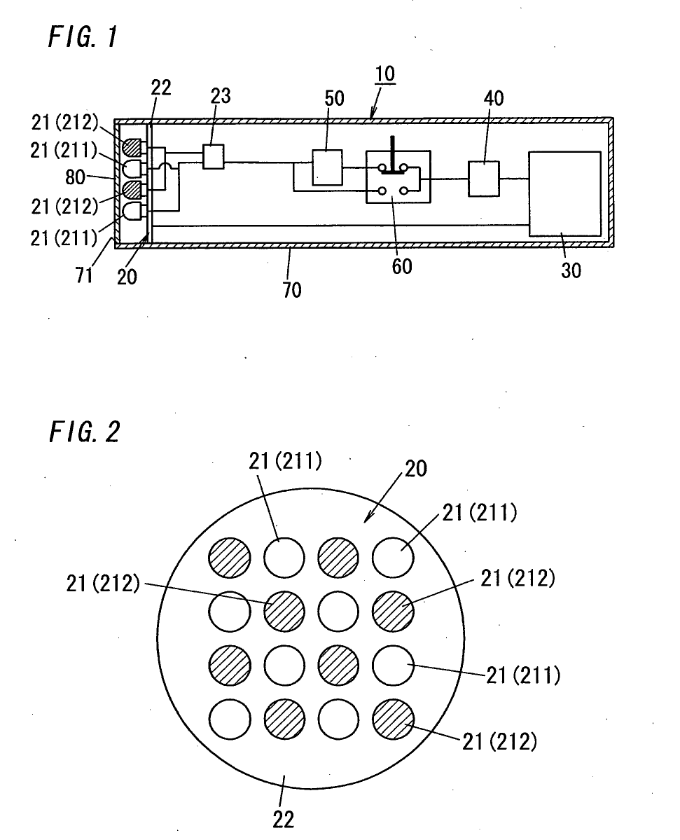

As shown in FIG. 1, the hair-growth device 10 of one embodiment of the present invention includes an irradiator 20, a power source 30, a timer 40, a pulse generator 50, a switching unit 60, a housing 70, and a cover 80.

As shown in FIG. 2, the irradiator 20 includes a plurality (eight, in the illustrated instance) of first light sources 211 and a plurality (eight, in the illustrated instance) of second light sources 212. Besides, when the first light source 211 does not need to be distinguished from the second light source 212, the first light source 211 and the second light source 212 are referred to as a light source 21, as necessary. In FIGS. 1 and 2, in order to distinguish the first light source 211 from the second light source 212, the second light source 212 is illustrated with hatching.

The light source 21 is a light emitting diode. The first light source 211 is configured to emit light (hereinafter referred to as “first light”, as necessary) having a wavelength of 950 nm. The second light source 212 is configured to emit light (hereinafter referred to as “second light”, as necessary) having a wavelength of 1450 nm.

The first and second lights have wavelengths equivalent to specific absorption wavelengths of water. The specific absorption wavelength of water is defined as a wavelength which shows a strong absorption of light (remarkable change in light absorption) relative to other wavelengths when the light is absorbed in the water. In other words, the specific absorption wavelength of water is defined as a wavelength at a peak of an absorption spectrum of water. The specific absorption wavelength is thought to result from an O—H combination tone of water. Especially, the specific absorption wavelength in a near-infrared region is one of 950 nm, 1150 nm, 1450 nm, and 1790 nm, for example. Besides, the peak of the absorption spectrum of water has a width. Therefore, a wavelength of the light emitted from the irradiator 20 is not strictly equivalent to the specific absorption wavelength of water. In other words, a wavelength of the light emitted from the irradiator 20 may be in a range regarded as being equivalent to the specific absorption wavelength.

The light source 21 is mounted on a substrate 22. As shown in FIG. 2, the substrate 22 is shaped into a circular disk shape. The eight first light sources 211 and the eight second light sources 212 are mounted on the substrate 22. For example, the light sources 21 are mounted on in a four by four matrix manner. In this situation, the first light sources 211 and the second light sources 212 are arranged alternately.

The irradiator 20 includes a controller 23 configured to control the first light sources 211 and the second light sources 212. For example, the controller 23 includes a first switch (not shown) interposed between the power source 30 and the first light sources 211, a second switch (not shown) interposed between the power source 30 and the second light sources 212, and a control circuit (not shown) configured to control the respective switches. Therefore, when the first switch is turned on, power is supplied from the power source 30 to the first light sources 211, thereby turning on the first light sources 211. When the second switch is turned on, power is supplied from the power source 30 to the second light sources 212, thereby turning on the second light sources 212. For example, the switches are semiconductor switching devices such as transistors, and the control circuit is a CPU or a logical circuit.

The controller 23 has first to fifth operation modes. In the first operation mode, the control circuit keeps turning off the both switches. In this situation, both the first light sources 211 and the second light sources 212 are kept turned off. In the second operation mode, the control circuit keeps turning on only the first switch. In this situation, only the first light sources 211 are kept turned on. In the third operation mode, the control circuit keeps turning on only the second switch. In this situation, only the second light sources 212 are kept turned on. In the fourth operation mode, the control circuit keeps turning on both the first switch and the second switch. In this situation, both the first light sources 211 and the second light sources 212 are kept turned on. In the fifth operation mode, the control circuit turns on the first switch and the second switch alternately. In this situation, the first light sources 211 and the second light sources 212 are turned on alternately.

As seen from the above, the controller 23 is configured to turn on the first light sources 211 and the second light sources 212 simultaneously and is configured to turn on the first light sources 211 and the second light sources 212 alternately.

The controller 23 includes a first operation unit (not shown) for manually switching among the five operation modes. The first operation unit is mounted on an outer surface of the housing 70.

The power source 30 is configured to supply power for turning on the light source 21 to the irradiator 20. For example, the power source 30 is a DC source configured to output a constant output voltage. A primary cell or a secondary cell can be adopted as the power source 30. Alternately, the power source 30 may have a function of converting an AC voltage received from a commercial power source into a DC voltage.

The timer 40, the switching unit 60, and the pulse generator 50 are interposed between the power source 30 and the irradiator 20.

The timer 40 is configured to break electrical connection between the power source 30 and the irradiator 20 when a predetermined time passes, for example. The timer 40 is defined as a time controller configured to allow the irradiator 20 to emit continuously light for at least 15 minutes.

The pulse generator 50 is configured to convert the output voltage of the power source 30 into a pulse voltage. In the present embodiment, the pulse voltage generated by the pulse generator 50 has a period of 500 Hz. The period of the pulse voltage is not limited to 500 Hz.

The switching unit 60 is configured to select an electric path for connection of the power source 30 and the controller 23 from a first electric path passing through the pulse generator 50 and a second electric path not passing through the pulse generator 50. In the first power path, the pulse generator 50 is inserted into the electric path between the power source 30 and the controller 23. In this situation, the pulse voltage is given to the irradiator 20. Therefore, the light source 21 emits light intermittently (pulse lighting). In the second power path, the pulse generator 50 is not inserted into the electric path between the power source 30 and the controller 23. In this situation, the constant output voltage is given to the irradiator 20. Therefore, the light source 21 emits light continuously (continuation lighting).

In brief, the switching unit 60 is configured to switch between the pulse lighting and the continuation lighting. The switching unit 60 includes a second operation unit (not shown) for switching manually between the pulse lighting and the continuation lighting. The second operation unit is mounted on the outer surface of the housing 70.

The housing 70 is configured to house the irradiator 20, the power source 30, the timer 40, the pulse generator 50, and the switching unit 60. The housing 70 is shaped into a circular cylindrical shape. The housing 70 is provided in its first axial surface (left surface, in FIG. 1) with a window 71 for allowing light from the irradiator 20 to go outside. The irradiator 20 is housed in the housing 70 such that the respective light sources 21 are visible via the window 71. The housing 70 has a diameter of 40 mm, for example.

The cover 80 is made of a translucent material (e.g. a glass and a translucent resin) and is shaped into a circular disk shape. The cover 80 has enough dimensions to cover the window 71. The cover 80 is fitted into the window 71. The cover 80 is used for protection of the irradiator 20. Besides, the cover 80 may be constructed to function as lens, as necessary.

A following first experimentation was performed in order to confirm hair-growth effect resulting from the aforementioned hair-growth device 10.

In the first experimentation, three observed areas are provided to lower limbs of respective subjects A and B. Each of the observed areas is a square area of 4 cm by 4 cm. Hairs sprouted on the observed area were shaved prior to beginning the first experimentation. Two of the three observed areas were used as irradiation areas, and the other one was used as a control area (reference area). By use of the hair-growth device 10, the first irradiation area was irradiated with light having a wavelength of 950 nm, and the second irradiation area was irradiated with light having a wavelength of 1450 nm. Additionally, in the first experimentation, the respective light sources 21 were turned on in the pulse lighting manner (at a frequency of 500 Hz). The control area was not irradiated with light from the hair-growth device 10.

Under the aforementioned condition, the irradiation area was irradiated with light continuously for 20 minutes every day over 5 consecutive days (20 minutes irradiation of light per day). A day next to a day when the irradiation is made first was defined as the first day. The observed areas were observed every 10 days from the first day.

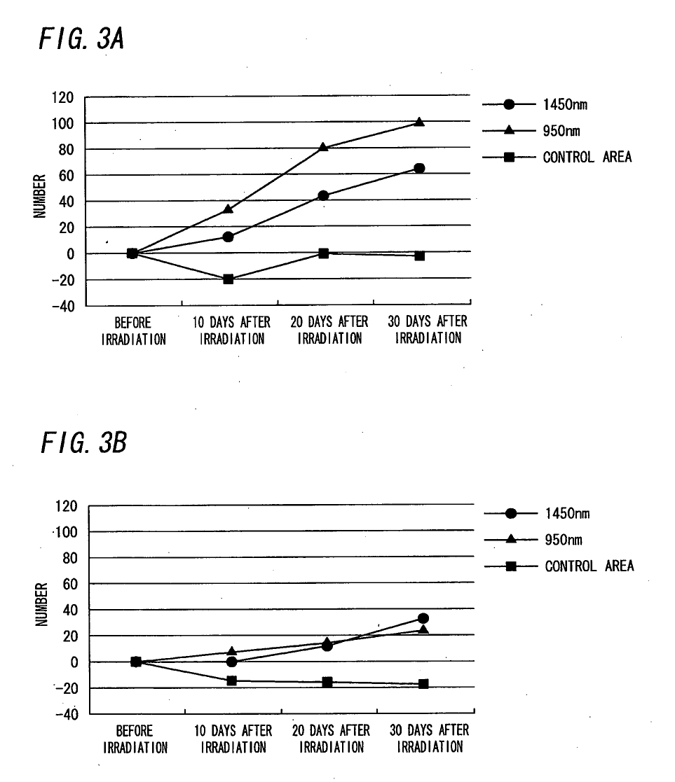

A result of the first experimentation is shown in FIGS. 3 to 5. Respective FIGS. 3A, 4A, and 5A show a result of the first experimentation with respect to the subject A, and respective FIGS. 3B, 4B, and 5B show a result of the first experimentation with respect to the subject B.

Besides, an analysis of the result of the first experimentation was made by performing image processing of photos of the observed areas by use of a personal computer. In the above analysis, the number of hairs, a thickness of a hair, and a length of a hair in the observed area were evaluated. The thickness of a hair is defined as a thickness (diameter) of a base portion (i.e., a portion of a hair outside of the skin and nearest thereto).

FIGS. 3A and 3B are graphs showing a transition of the number of hairs with respect to elapsed days. A horizontal axis represents the elapsed days, and a vertical axis represents a variation of the number of hairs relative to a first initial value (the number of hairs just after being shaved).

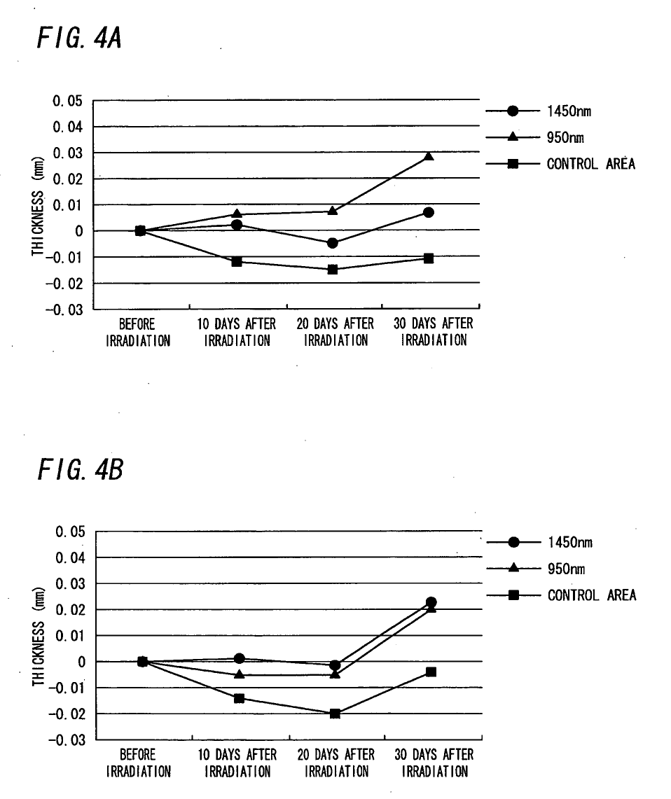

FIGS. 4A and 4B are graphs showing a transition of the thickness of a hair with respect to elapsed days. A horizontal axis represents the elapsed days, and a vertical axis represents a variation of an average of the thickness of a hair relative to a second initial value (average of the thickness of a hair just after being shaved).

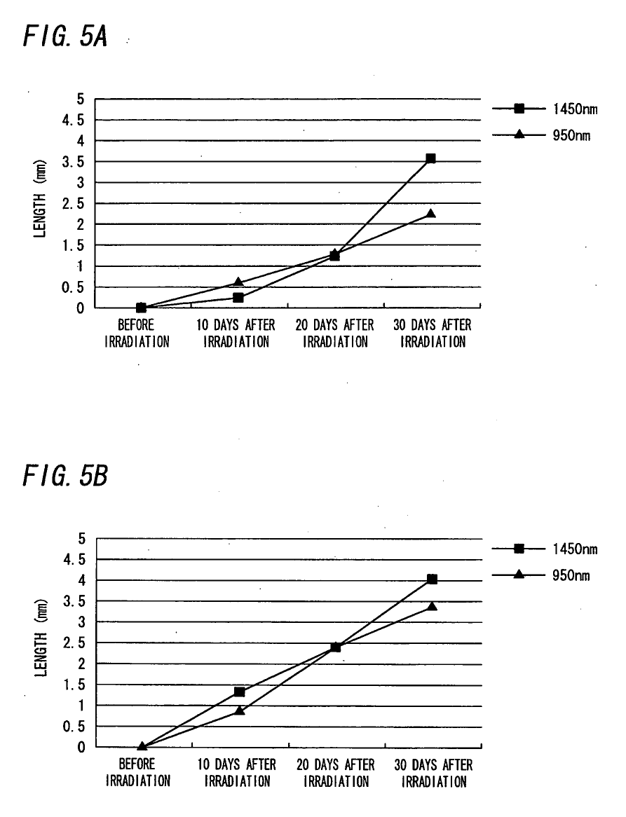

FIGS. 5A and 5B are graphs showing a transition of the length of a hair with respect to elapsed days. A horizontal axis represents the elapsed days, and a vertical axis represents a variation of the length of a hair relative to a third initial value (the length of a hair just after being shaved).

As shown in FIGS. 5A and 5B, results at 30 days after the irradiation with respect to both the subjects A and B show that the length of a hair of the second area was greater than that of the first area. That is, it indicated that, concerning the length of a hair, the second light produces superior hair-growth effect on both the subjects A and B in comparison with the first light.

As shown in FIGS. 3 and 4, results with respect to both the subjects A and B show that a difference between the control area and the irradiation areas appeared clearly.

FIG. 3A shows a result at 30 days after the irradiation with regard to the subject A that the number of hairs of the first irradiation area increases by as much as 100 times relative to the control area, and the number of hairs of the second irradiation area increases by as much as 60 times relative to that of the control area. FIG. 4A shows a result at 30 days after the irradiation that the thickness of a hair of the first irradiation area increases by as much as 0.04 mm relative to the control area, and the thickness of a hair of the second irradiation area increases by as much as 0.02 mm relative to that of the control area.

FIG. 3B shows a result at 30 days after the irradiation with regard to the subject B that the number of hairs of the first irradiation area increases by as much as 40 times relative to the control area, and the number of hairs of the second irradiation area increases by as much as 50 times relative to that of the control area. FIG. 4B shows a result at 30 days after the irradiation that the thickness of a hair of the first irradiation area increases by as much as 0.025 mm relative to the control area, and the thickness of a hair of the second irradiation area increases by as much as 0.03 mm relative to that of the control area.

The result of the first experimentation showed that growth of hair was promoted by irradiating skin with light having a wavelength equivalent to the specific absorption wavelength of water with relation to both the subjects A and B.

As apparent form the result of the first experimentation, a superior hair-growth effect can be obtained by irradiation a living body with light having a wavelength equivalent to the specific absorption wavelength of water. This could be explained by the following reason. The living body shows an efficient absorption reaction which results from an O—H combination tone of water when irradiated with the light of a wavelength equivalent to the specific absorption wavelength. In other words, the living body is activated by irradiation of light having the wavelength equivalent to the specific absorption wavelength. When the living body is so activated, the hair-growth is promoted.

Additionally, after the first experimentation, no skin disorders and no inflammation were not observed in the respective irradiation areas. This shows that light having a wavelength equivalent to the specific absorption wavelength of water does not cause abnormalities of a skin (flesh). Besides, the light source 21 was turned on in the pulse lighting manner in the first experimentation. When the light source 21 was turned on in the continuous lighting manner, a result similar to that of the first experimentation was obtained.

The result of the first experimentation shows that light having a wavelength of 950 nm or 1450 nm produces the distinguished hair-growth effect. Further, experimentation similar to the first experimentation was performed for light having a wavelength of 1150 nm and light having a wavelength of 1790 nm. This result shows that light having a wavelength of 1150 nm or 1790 nm produces the distinguished hair-growth effect in similar fashion as light having a wavelength of 950 nm or 1450 nm and did not cause inflammation of a skin. That is, light having a wavelength equivalent to the specific absorption wavelength of water produces hair-growth effect without injuring a skin.

The aforementioned hair-growth device 10 promotes the hair growth through a mechanism of that the light of the wavelength equivalent to the specific absorption wavelength of water is absorbed into a light absorption component already existing in the skin. The light absorption component absorbs more efficiently the light having a wavelength equivalent to the specific absorption wavelength rather than other light having a wavelength different from the specific absorption wavelength. Therefore, in contrast to the irradiation of light having a wavelength different from the specific absorption wavelength, the living body is activated to a greater extent by irradiation of the light having a wavelength equivalent to the specific absorption wavelength, and sees promotion of hair-growth.

As seen apparent from the above, the hair-growth device 10 can promote growth of hairs. Therefore, it is possible to provide hair-growth effect without using a medical agent. Additionally, in contrast to light having a wavelength of 890 nm, the light having a wavelength equivalent to the specific absorption wavelength of water does not irritate a skin or skin inner tissue. Therefore, it is possible to grow a user's hair in a comfortable manner without injuring a skin.

According to the preferred embodiment, it is possible to emit the light having a wavelength of 950 nm or 1450 nm, which is equivalent to the specific absorption wavelength of water and produces an excellent effect for improvement of the hair-growth effect. Besides, light emitted from the irradiator 20 may have a wavelength of 1150 nm or 1790 nm.

The result at 30 days after the irradiation with regard to the subject A shows that the first irradiation area was greater in the number of hairs and the thickness of a hair than the second irradiation area (see FIG. 3A and FIG. 4A).

By contrast, the result at 30 days after the irradiation with regard to the subject B shows that the second irradiation area was greater in the number of hairs and the thickness of a hair than the first irradiation area (see FIG. 3B and FIG. 4B).

As seen from the above, a comparison of the first irradiation area and the second irradiation area shows that the first light produces the superior hair-growth effect on the subject A than the second light, and that the second light produces the superior hair-growth effect on the subject B than the first light. Consequently, a wavelength of light optimal for hair-growth differs depending on a condition of a subject.

The subject A was a man in his twenties, and the subject B was a man in his fifties.

The result of the first experimentation shows that a wavelength of light optimal for hair-growth is different according to a user's age (that is, an age of a person to be irradiated with light affects the hair-growth effect).

In the fourth operation mode, the controller 23 turns on both the first light sources 211 and the second light sources 212 simultaneously. In the fifth operation mode, the controller 23 turns on the first light sources 211 and the second light sources 212 alternately.

Accordingly, the hair-growth device 10 can emit the first light of a wavelength of 950 nm suitable for a man in his twenties and the second light of a wavelength of 1450 nm suitable for a man in his fifties simultaneously or alternately.

Therefore, even if a wavelength of light optimal for hair-growth is varied depending on a condition (age) of a person to be irradiated with light, it is possible to promote efficiently and successfully hair-growth. That is, it is possible to provide the stable hair-growth effect irrespective of an age of a person to be irradiated with light.

Besides, the controller 23 has the second operation mode where only the first light sources 211 are kept turned on, and the third operation mode where only the second light sources 212 are kept turned on. Therefore, the light source 21 to be used can be selected in accordance with a user's age. Thus, it is possible to provide an optimal hair-growth effect according to a user's age.

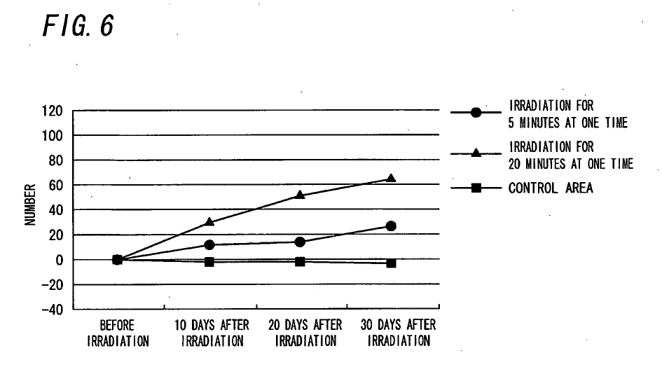

FIG. 6 shows a result of a second experimentation performed in order to confirm effect of the hair-growth device 10 of the present embodiment.

In the second experimentation, three observed areas are provided in a similar manner as the first experimentation. Two of the three observed areas were used as irradiation areas (third irradiation area and fourth irradiation area), and the other one was used as a control area (reference area). Both the third irradiation area and the fourth irradiation area were irradiated with light having a wavelength of 950 nm by turning on the first light sources 211 in the pulse lighting manner at a frequency of 500 Hz. Besides, the control area was not irradiated with light from the hair-growth device 10.

Under the aforementioned condition, the third irradiation area was irradiated with light continuously for 20 minutes every day over 5 consecutive days (20 minutes irradiation of light per day). The fourth irradiation area was irradiated with light continuously for 5 minutes every day over 5 consecutive days (5 minutes irradiation of light per day). A day next to a day when the irradiation is made first was defined as the first day. The observed areas were observed every 10 days from the first day.

As shown in FIG. 6, both the third and fourth irradiation areas produced a higher hair-growth effect than the control area. Especially, the third area produced a higher hair-growth effect than the fourth irradiation area.

Further, skin disorders and inflammation were not observed in both the third and fourth irradiation areas. In the third and fourth irradiation areas, abnormalities of a skin did not occur. Besides, the first light sources 211 were turned on in the pulse lighting manner in the second experimentation. When the first light sources 211 were turned on in the continuous lighting manner, an effect similar to that of the second experimentation was obtained.

The result of the second experimentation shows that the hair-growth effect is enhanced as the continuous irradiation time of the light is increased. In the prior hair-growth method, the length of the continuous irradiation time of light is limited because light causes inflammation. In contrast, the hair-growth device 10 emits light having a wavelength equivalent to the specific absorption wavelength of water. Therefore, even if the continuous irradiation time of light increases, the skin does not suffer from inflammation or the like. That is, according to the hair-growth device 10, it is possible to irradiate a skin with light for a long time without causing burn injury and inflammation in the skin.

Experimentations having the different continuous irradiation time were performed in a similar manner as the second experimentation. Results of these experimentations show that a superior hair-growth effect is observed when the continuous irradiation time is 15 minutes or more.

Therefore, the timer 40 is preferred to be configured to allow the irradiator 20 to emit light for at least 15 minutes. In this situation, a superior hair-growth effect can be obtained.

Besides, the light source 21 may be selected from an organic electroluminescent device, a discharge lamp, and a light bulb. In brief, the light source 21 may be configured to emit light having a wavelength equivalent to the specific absorption wavelength of water. For example, the first light sources 211 and the second light sources 212 may be arranged per groups each including the light sources having the same wavelength or arranged without being grouped by the wavelength. The first light sources 211 and the second light sources 212 need not be arranged at regular intervals. The number of the first light sources 211 need not be equivalent to the number of the second light sources 212. The wavelength of the light from the irradiator 20 is not limited to the aforementioned instance, and may be selected from the specific absorption wavelength of water. The irradiator 20 may include the three or more types of the light sources 21 having wavelengths different from each other. The irradiator 20 may include only one type of the light sources 21.

Besides, in the hair-growth device 10, the timer 40, the pulse generator 50, and the switching unit 60 are optional. Additionally, in the controller 23, the fourth operation mode and the fifth operation mode are optional.

Next, an explanation is made to a hair-growth method of one embodiment of the present invention. The hair-growth method of the present embodiment includes a step of irradiating a skin with light having a wavelength equivalent to the specific absorption wavelength of water. According to the hair-growth method, it is possible to provide hair-growth effect without using a medical agent. Additionally, the light has a wavelength equivalent to the specific absorption wavelength of water. Therefore, the light does not irritate a skin or skin inner tissue. Thus, it is possible to grow a user's hair in a comfortable manner without injuring a skin.

In addition, the light emitted to the skin is preferred to be light having a wavelength of 950 nm or 1450 nm. In this situation, it is possible to emit the light having a wavelength of 950 nm or 1450 nm, which is equivalent to the specific absorption wavelength of water and produces an excellent effect for improvement of the hair-growth effect.

Preferably, in the step of irradiating the skin with the light, the first light having a wavelength of 950 nm and the second light having a wavelength of 1450 nm are emitted simultaneously or alternately. In this situation, the first light and the second light are used in combination to give the different hair-growth effects depending on an age of person to be irradiated with light. Therefore, it is possible to provide the stable hair-growth effect.

Preferably, the light is emitted to the skin continuously for at least 15 minutes. In this situation, it is possible to improve the hair-growth effect.

Preferably, the wavelength of the light emitted to the skin is selected on the basis of an age of a person to be irradiated with light. In this situation, it is possible to provide the hair-growth effect optimal for the age.

1. A hair-growth device comprising an irradiator configured to irradiate one's skin with light having a wavelength equivalent to a specific absorption wavelength of water.

2. A hair-growth device as set forth in claim 1, wherein
the light has a wavelength of 950 nm or 1450 nm.

3. A hair-growth device as set forth in claim 1, wherein
said irradiator includes a first light source configured to emit light having a wavelength of 950 nm, a second light source configured to emit light having a wavelength of 1450 nm, and a controller configured to control said first light source and said second light source,
said controller being configured to turn on said first light source and said second light source simultaneously or alternately.

4. A hair-growth device as set forth in claim 1, wherein
said hair-growth device includes a time controller configured to allow said irradiator to emit continuously the light for at least 15 minutes.

5. A hair-growth method comprising a step of irradiating a skin with light having a wavelength equivalent to a specific absorption wavelength of water.

6. A hair-growth method as set forth in claim 5, wherein
the light emitted to the skin has a wavelength of 950 nm or 1450 nm.

7. A hair-growth method as set forth in claim 5, wherein
first light having a wavelength of 950 nm and second light having a wavelength of 1450 nm are emitted simultaneously or alternately.

8. A hair-growth method as set forth in claim 5, wherein
the light is emitted to the skin continuously for at least 15 minutes.

9. A hair-growth method as set forth in claim 5, wherein
said method comprises a step of selecting the wavelength of the light emitted to the skin on the basis of an age of a person to be irradiated with the light.
